(19) United States
(12) Patent Application Publication
(10) Pub. No.: US 2002/0114728 A1
(43) Pub. Date: Aug. 22, 2002

(54) Electronic Sterilizer

(76) Inventors: Victor V. Kulish, Sumy (UA); Alexandra C. Melnyk, Powell, OH (US); Volodymryr A. Khvorost, Sumy (UA)

Correspondence Address: Gerald L. Smith, Mueller and Smith, LPA, 7700 Rivers Edge Drive, Columbus, OH, 43235, US

(21) Appl. No.: 10/022,288

(22) Filed: Dec. 20, 2001

(30) Foreign Application Priority Data
Feb. 13, 2001 (UA) 2001020953

(51) Int. Cl. A61L 2/00; A61L 9/00; A62B 7/08; B01J 19/08

(52) U.S. Cl. 422/22; 422/32; 422/4; 422/121; 422/186

(57) ABSTRACT

1. The Object of the invention: electron sterilizer

2. The application Branch: the invention enables its application as a commercial-type compact electron sterilizer for sterilization of food products, medical and biological preparations, medical and biological equipment, and also for disinfecting of water, including the waste waters, agricultural production products (including meat, milk, grain, beans) etc. and is designed to destroy (or inhibit) pathogenic bacteria, viruses, parasites, and fungi which are present in the objects of treatment.

3. The substance of the invention: The sterilizer consists of a multi-channel linear induction accelerator (MLIA) 1, with attached to it a block 2 of the outlet devices, and with an irradiation system block 3 attached to the outlet-device block 2. The transport system 4, on which the irradiation (treatment) objects 5 are placed, is positioned under the block 3. The ventilation system 6 is positioned in the way that allows isolation of the irradiation system 3 and transport system 4, and the working field where sterilization takes place from remaining structural elements of MLIA. The lower protection system 7 is placed under the transport system 4 while the upper protection system is placed above the accelerator 1 and the transport system 4.

4. The alternative realization: electron sterilizer based on a single-channel induction accelerator.

5. Technical advantage: An increase of productivity, compactness, and electromagnetic compatibility, and a technological possibility (in other words, technologic adequacy for the conditions typical for agricultural, food-production and pharmaceutical industries), and an increase of safety of exploitation, besides a decrease of the manufacturing and ex-exploitation costs and overall simplification of the (sterilizer's) structure.

### BACKGROUND OF THE INVENTION

The invention corresponds to the instruments for treatment of the products of the agricultural, food industry, and pharmaceutical industry and also as instrument for disinfect or decontaminate various objects, especially in the sterilization technique, and can be used as a commercial instrument for the sterilization of the food products, medical and biological preparations, decontamination of meats, water, grains, fruits, products of populated points of the vital activity, and animal farms.

There is known a device which is able to work as an electronic sterilize [Sculer R. E. Radiation sources—‘E’B//Radiat. Phys. Chem., vol. 14,1979, pp. 171-184]. It consists from a source of a relativistic electron beam, an outlet device (attachment), an irradiation and a transport systems. An electrostatic electron accelerator (EEA) serves as the source of the relativistic electron beam. An excessive overall size, a high cost of its production and its service, and an excessive danger of its operation are the basic shortcomings of this device. All the above mentioned shortcomings are caused by the peculiarities of the electrostatic accelerator construction since the accelerator represents at least 80% of the total overall size of the sterilizer. This is explained primarily by the presence of the high-energy potential (from hundreds of kV to tens of Mv). Special electrotechnical oils and high-pressure gases ( from 5 to 30 atm.) are used for the protection of the isolation of the elements which exist under high voltage. These shortcomings are the basic obstacle for the production of compact, relatively inexpensive, and safe in exploitation sterilizers. These shortcomings are especially essential when working with the electron beams stronger than 1 Me.

The known device, which can work like an electron sterilizer [McKeown J. Technology review of accelerator facilities.//Radiat. Phys. Chem., vol. 35, No. 4-6, 1990, pp. 606-611]. This device consists also from a source of a relativistic electron beam, output attachment, and the irradiation and the transport systems. The basic difference of this device from the previously described analogue is the formation of the source of the relativistic electron beam in the form of a linear radio-frequency accelerator (LRFA). This allows a decrease of the overall dimensions of the system, especially when working with beams of higher that 1 MeV. This relative decrease in the dimensions is achieved by a significant complication of construction, and by an increase of its price.

The latter is related, first of all, with the super-powerful microwave generators (MWG) (clystrons and magnetrons) serving as the energy source of the radio-frequency accelerator. These generators are expensive in production as well as in operation. For instance, the serial sterilizer based on LRFA costs today from 0.8 to 3.0 million USA dollars. In addition to this, clystrons (magnetrons) which are used today require systematic changes in the exploitation process, which in turn is related with the technologic limitations in their service. Since these sterilizers belong to the high-complexity electronic systems they can be served only by a group of highly qualified specialists.

The following is the consequence of using the MWG generators in this type sterilizers. A transition from the use of the direct acceleration method by the super-high electrostatic fields (SHEF), which was used in the first analogue, to the acceleration method by MWG, which was used in the second analogue, allows a decrease of the danger which is related with the presence of the super-high electrostatic voltage. However, simultaneously appeared a new danger for the service personnel caused by irradiation with powerful microwaves. Consequently, the average potential level of danger in using such systems in civilian branch of production, such as food or pharmaceutical, etc. industry, is unacceptably high.

There is also know a device which can be used as sterilizer [Scarpetti R. D., Boyd J. K., Earley G. G., ea. “Upgrades to the LLNL flash X-ray induction linear accelerator (FXR)” Digest of Technical Papers of 11^{th} IEEE International Pulsed Power Conf. Baltimore, Md. USA, Jun. 29-Jul. 2, 1997, v. 2, pp 597-602, 1998]. Like the first and the second analogue, this device consists from a source of the relativistic electron beam, output attachment, and the irradiation and the transport systems. Its peculiarity consists of the source of the relativistic electron beam, which is made in a form of the linear induction accelerator (LIA). Acceleration of electrons in LIA is achieved by the action of the longitudinal vortex electron field, having relatively low frequency (tenths Mhz), which is generated in the working acceleration channel by special inductors. In contrast to the field generated beyond the working channel of the accelerator, this field is called internal. By using the vortex electric fields for acceleration, a large part of the above mentioned shortcomings is removed in the sterilizers, built on the EEA and LRA basis. Especially LIA has a simple structure, significantly lower price, higher safety, and a simple operability. The basic shortcomings of the LIA sterilizers is the excessive linear working dimensions, which in turn, are caused by the marked linear dimensions of the LIA, and the necessity to secure the significant distance between the outcome, and the object of its irradiation. For instance, the length of the LIA, described in the work of Scarpetti et. al [Scarpetti R. D., Boyd J. K., Earley G. G., et al. “Upgrades to the LLNL flash X-ray induction linear accelerator, (FXR), “Digest of Technical Papers of 11ₜₕ IEEE International Pulsed Power Conf., Baltimore, Md., USA, Jun. 29-Jul. 2, 1997 v. 2, pp 597-602, 1998], at approx. 18 MeV energy is approx. 20 meter. Besides this, a significantly strong vortex external field is generated in the space surrounding a working LIA. This field might have a negative effect on the working personnel, as well as on the surrounding apparatuses, leading to the formation of a peculiar “dead zone” around the accelerator. The latter has two basic negative effects: 1. It requires to secure an additional space around the sterilizer. This space cannot be used by the attending personnel or the accompanying apparatuses without a special protective screening. 2. As a rule, the irradiation of the sterilizing objects by a strong electric field is forbidden, because it changes the nutritional characteristics in non-foreseen directions or it might destroy the objects of the irradiation (e. g. ampoules with liquid pharmaceutical preparations, liquid foodstuff such as flask or can of beer, etc.). To eliminate this situation, a significant distance has to be secured between the LIA output and the irradiation system at which (distance) the intensity of the external electric field LIA will decrease to acceptable values. As a consequence, the sterilizers based on LIA are excessively large and do not fit to the technologic conditions, which are typical in treatment of the agricultural products or in food and pharmaceutical industry. In other words, under such conditions they are characterized by a low level technology and electromagnetic compatibility.

The low productivity is one more shortcoming of the LIA-based electron sterilizer. This is related with the fact that for the sterilization only one limited-width and limited-current strength electron beam is used. Since the exposure time (time of irradiation) of the treated objects is limited by the beam-current strength as well by the dimensions of the irradiation zone, in a case of a single working beam, the speed of the transporting belt (which equals the irradiation zone mobility on the surface of an irradiation object) is low. Tens of seconds to single minutes time are practically needed for irradiation of one object. On the other hand, e.g. in cases when the sterilizing effect is achieved by direct, electron beam irradiation of an object, there are technologic limitations for the beam-current strength. This also leads to a necessary increase of the irradiation time and to a limitation of the productivity of the sterilizer. Consequently, the above mentioned factors reveal that the presence of the LIA-based single electron beam is responsible for the limitation of the productivity of the LIA-based sterilizer.

Hence, low productivity, large working dimensions, high price of manufacturing and operation, non-technologic nature, low electromagnetic compatibility, and high complexity and degree of the danger in their exploitation are the basic shortcomings of the LIA-based sterilizers. Because of this the LIA-based sterilizers are commercially not applicable and are used only in some special research programs [e.g. Scarpetti R. D., Boyd J. K., Earley G. G., et al. “Upgrades to: the, LLNL flash X-ray induction linear accelerator (FXR), “Digest of technical papers of 11^{th} IEEE International Pulsed Power Conf, Baltimore, Md. USA, Jun. 29-Jul. 2, 1997, v. 2, pp. 597-602, 1998]. This device resembles most the proposed invention, considering the technical nature and result, which are obtained and considered as the prototype.

### BRIEF SUMMARY OF THE INVENTION

The aim of the invention is manufacture of a commercial-type electron sterilizer which has a high productivity, is relatively compact, safe in exploitation, inexpensive to manufacture and to use, has a simple construction, high electromagnetic compatibility and technology (in other words, with technology which is adequate in the conditions which are found in the civil industry branches such as agricultural product and food processing, and in pharmaceutical industry).

This task is solved by using the source of the relativistic electron beams in the form of a multi-channel linear induction acceleration in the electron sterilizer which is composed of the source of the relativistic electron beams, and output and irradiation and transport systems. Seven structural variants and their combinations are considered in manufacturing such sterilizers.

The first variant, has multi-channel, linear induction accelerator as the source of the individual relativistic electron beam.

In the second variant, the multi-channel linear induction accelerator consists as a source of at least two relativistic electron beams.

In the third variant, the irradiation block system is made in a form of electron-beam scanning system, with each beam connected to one of the output of the output-device block, which in turn is made as a vacuum window allowing an electron beam to exit from the vacuum into the air atmosphere.

In the fourth variant, the irradiation block system is made in the form of electron-beam defocusing system, each of which contacting one output of the output-lock attachment. Each of the outputs has a form of a vacuum window which allows exit of an electron beam from vacuum into the air atmosphere.

In the fifth variant, the irradiation system has a form of an X-ray target attached to the outputs of the output block of the system.

In the sixth variant, the irradiation is achieved in a form of scanning for each of the electron beams and the output window having a form of an X-ray target.

In the seventh variant, the irradiation-system block has the form of an electron-beams-defocusing system with the output window having a form of an X-ray target.

Manufacturing of the source of the relativistic electron beams in a form of a multi-channel linear induction accelerator (MLIA) allows to increase productivity, decrease working dimensions, decrease the costs of manufacturing and operation, increase the degree of safety and technology and electromagnetic compatibility, and secure the commercial effectiveness of practical use of the electron sterilizer.

The possibility of a simultaneous acceleration of many electron beams in a MLIA in one and the same direction allows realization of a multiple irradiation system of the treated (irradiation) objects by successively different beams without increasing intensity of each of these (partial) beams. Because of this, a significant shortening of the exposure time and, accordingly, an increase of the sterilizer's productivity can be achieved at the same (as compared with the prototype) current of each of the beams. Since, at the same acceleration characteristics as those of the prototype, the proposed sterilizers are smaller and are more safe since the MLIA have much lower intensity of the external field and, respectively, almost two times higher intensity of the internal (accelerating) field, at the same acceleration, linear dimensions of the MLIA are essentially smaller than that of the linear dimensions of the LIA and the external field in MLIA is much (tens of thousands, depending on the distance from inductors) weaker than the external field of the prototype. Accordingly, in the proposed construction, the minimal acceptable distance between the accelerator and the irradiation system (the distance at which the safety of the effect of the external electric field on treated samples can be guaranteed) can be significantly smaller (a few times or a few tens times smaller). This means that the expense, related with the safety and the protection of the attending personnel and other systems and apparatuses of the sterilizer from the undesirable effect of the electric field can be decreased. The costs of the manufacturing of the sterilizer and on the routine maintenance as well as the danger of an electric breakdown in the system are decreased. As the result of it, the safety, electromagnetic compatibility and the technology of the proposed electron sterilizer are increased. Consequently, practical application of the foreseen discovery in civil branches of industry (e.g. in agricultural, pharmaceutical, or food industries, etc.) becomes commercially expedient.

Patent search process did not reveal any information about the essential characteristics of the invention and the effects of the invention on the achieved results. This allows to derive a conclusion that the proposed technical solution corresponds to the patentability criteria “novelty” and “the invention level”.

### BRIEF DESCRIPTION OF THE DRAWINGS

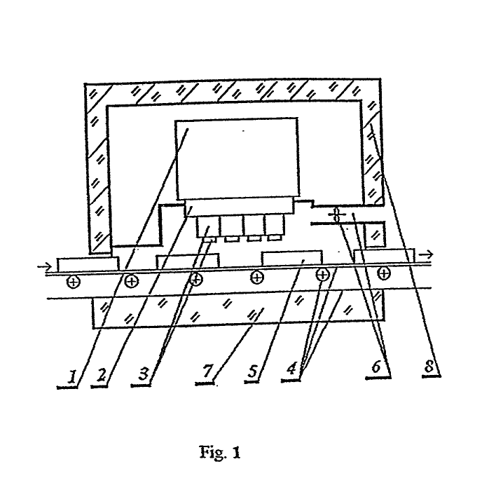

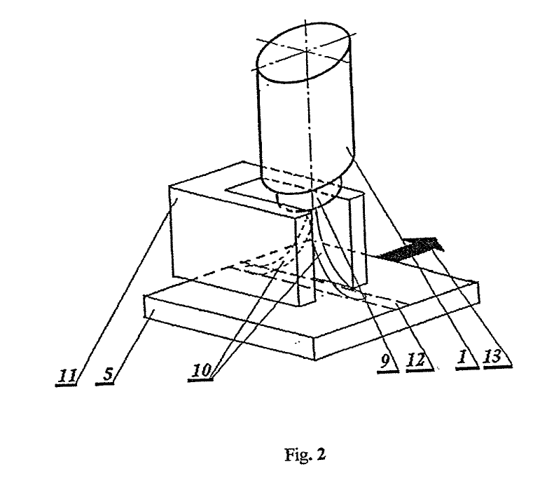

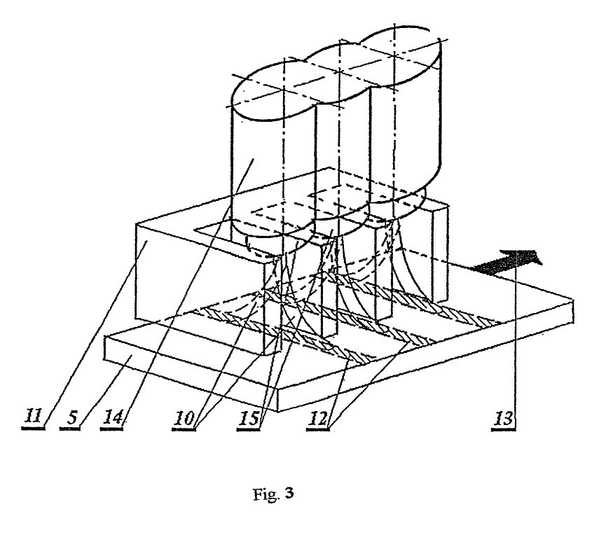

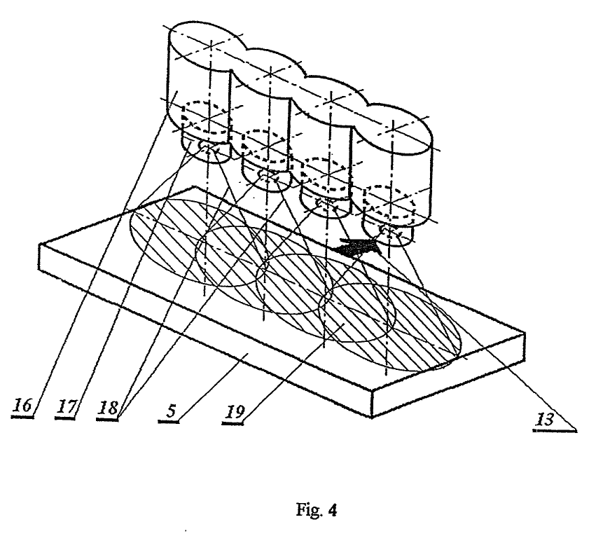

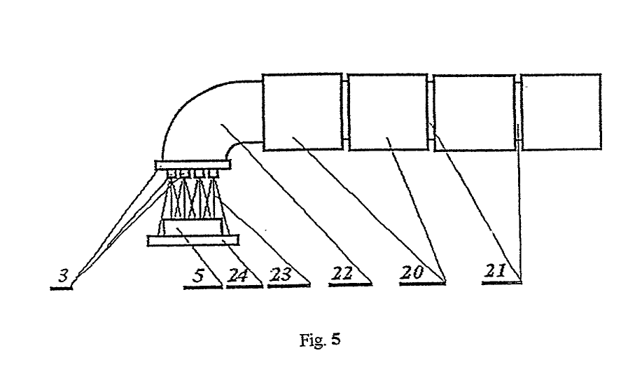

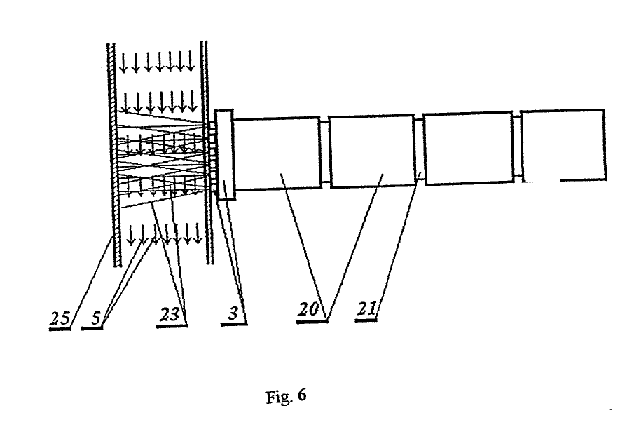

The essence of the invention is explained by illustrations, where FIG. 1 shows the overall construction of the electron sterilizer; FIG. 2 illustrates the variant of the sterilizer with one working electron beam and the system of its scanning; FIG. 3 illustrates the variant of the electron sterilizer construction with a few working electron beams and a system of its scanning; FIG. 4 illustrates the electron sterilizer construction with a few electron beams and a system of their defocusing; FIG. 5 illustrates a variant of the electron sterilizer structure with horizontal position of the MLIA and the transport system, and FIG. 6 illustrated the variant of the electron sterilizer with an horizontal position of MLIA but a vertical position of the transport system.

### DETAILED DESCRIPTION

In FIG. 1, 1 represents the MLIA. A block of the output-devise block 2 is attached to its output. The irradiation system 3 is attached to the output devise block 2. The transport system 4, where the sterilization of objects occurs, is located directly under the irradiation system 3. The ventilation system 6 is placed in such way that the irradiation system 3 and the working field of the irradiation on the transport system 4 (where the sterilization of the objects takes place) are isolated from the rest of the structural elements of the MLIA. A lower protection shield 7 is placed under the transport system 4 when the upper protection shield 8 is placed above the accelerator 1 and the transport system 4.

The FIG. 2, illustrates the variant of an electron sterilizer structure with one working electron beam and corresponding one scanning system. Here, the output attachment block 2 consists from only one output devise 9, which is attached to the output of MLIA 1. The output devise 9 has a form of a vacuum window which enables the transfer of the relativistic electron beam 10 from vacuum (which is realized within the accelerating channels MLIA 1) directly into the air atmosphere. The irradiation block 3 has only one irradiation system which has a form of a scanning system 11 of the electron beam 10. The irradiation object 5 is placed under the scanning system 11. The scanning angle of the beam 10 in the system. 11 is such that the irradiation. path 12 covers totally the transverse dimension of the irradiation object 5 (relatively to the direction of the irradiation object 5 on the transport system 4 which is designed by the arrow 13).

The FIG. 3, illustrates the variant of the electron sterilizer structure, which differs from the structure in the FIG. 2 only by that here the MLIA has the form of a 3-channel MLIA 14. The output attachment block 2 has three different output devices made in the form of vacuum windows 15. The irradiation system block is in the form of a triple scanning block 11.

The FIG. 4 illustrated the variant of the electron sterilizer structure in which MLIA has a form of the four-channel MLIA 16. In contrast to the structure variants presented above in FIG. 2 and FIG. 3, here the irradiation system 3 is made as a four-defocusing system block 17 of electron beams 18. The irradiation object 5 is located immediately under the defocusing-system block 17 resulting in the maximal dimension of the irradiation object covered by the irradiation path, which in the given case is presented as a superposition of irradiation spots 19. The irradiation is oriented perpendicularly to the irradiated object 5 movement direction 13 in the transport system 4.

The sterilizers are proposed having azimuthally-symmetric and azimuthally-asymmetric defocusing systems 17. In the first case the irradiation spots 19 on the irradiation objects 5 have circular form. In the second case, the irradiation spots have an ellipsoid, rectangular, or a more complex form. Simultaneously with this, the defocusing system has such structure and is located in such way that all the irradiation sports 19 of all electron beams 18 confluence on the irradiation object 5 into a continuous irradiation path.

The FIG. 5 illustrates a variant of an electron sterilizer with a horizontal position of MLIA 1 and of the transport system 4. Here MLIA 1 is composed of four sections 20, which are interconnected by transition devices 21. The transition device block 2 has a form of a block 22 of turning magnetic system. The irradiation block is attached to the turning magnetic system block 22. The electric beams 23 are directed on the irradiation object 5, which in turn is located horizontally on the oriented transport system 24.

The FIG. 6 illustrates the variant of the sterilizer structure with horizontally placed MLIA and vertically placed transport system 25, which has a form of a vertical transporter, turbo or pneumo-transporter. Here, like in FIG. 5, MLIA 1 consists of four sections 20, which are interconnected by transition devices 21. The irradiation block is positioned near the wall of the vertically oriented transport system (e.g. turbo-conductor, pneumo-conductor, etc.) 25, along which the irradiation object(s) 5 (liquids, powders, etc.) are moved vertically. The irradiation system 3 and the transport system 25 are constructed in such way that the electron beams 23 in the working volume of the transport system 25 form a continuous zone of irradiation.

Variants of the electron sterilizers are proposed in which the irradiation-system block 3 encloses the X-ray target block which is located at the outlet of the block 3. Three basic structures of such X-ray electron sterilizers are proposed.

In the first structure, the irradiation system block 3 has a form of the X-ray targets which are placed at the outlet of the block 2. Each target is constructed in such way that each X-ray beam which secures the generation of a diverging X-ray beam irradiation. Consequently, as it is illustrated by the structure in FIG. 4 of the electron sterilizer, the divergence angles are chosen in the way that all X-ray beams form a continuous irradiation path on the irradiation object.

In the second structure, the irradiation system block 3 has a form of the electron-beam scanning which are in contact with the X-ray targets at the cutlet.

In the third structure, the irradiation system block consists of an electron-beam defocusing block in which the outlet window has a form of an X-ray target.

The work of the electron sterilizer depends on the following: In the variant illustrated by FIG. 2, when the scanning system 11 is turned off, the electron beam 10 is moving vertically, forming, on the surface of the irradiation object 5 a spot, the form of which coincides with the form of the cross-section of the beam 10. As a rule, the dimensions of this spot are much smaller than the dimensions of the irradiation object 5. Consequently, in such case, only a small surface of the object 5 can be irradiated. When the scanning system 11 is turned on, a periodically alternating in time magnet field is generated in the process of crossing of the electron beam 10. It means that, in the horizontal plane, the alternating in time Lorentz force begins to act on the electric beam 10. Consequently, under the effect of Lorentz force, at each moment of time, the electrons of the beam 10 deviate from the previously strait-line direction of movement for an angle which depends at a given moment on the strength of the magnetic field as well as on the energy of the beam. Since, as it was mentioned before, the magnetic field changes with time, the deviation angle changes by the sign and by magnitude as function of time also. As a consequence, the electron beam 10 deviates systematically (it means scanning) from the vertical (line) in the plane perpendicular to the irradiation—object 5 plane, which is called the scanning plane. A peculiar path, which is called the irradiation path 12, forms on the surface of the irradiated object 5 within time interval which is much larger than the time of the change of the magnetic field. Since the irradiation objects 5 are shifted on the transport system 4, relatively to the MIA 1, the irradiation path 12 gradually moves along the irradiation object surface, parallel to itself. This assures the sterilization.

A known technologic process is proposed to use in order to achieve the sterilization effect at electronic sterilization. In case when the sterilization is achieved by the effect of an electron beam two processes are traditionally applied. The first is called electron sterilization and the second is known as a radiochemical sterilization. These technologies are proposed in this invention. The essence of the technologic processes lies in the following.

As it is known, the relativistic electron beams are able to penetrate in the depth of a treated material. The penetration depth depends mainly on energy of the electrons. For instance, 1 MeV electron beam is able penetrate 5 mm thick walls of a glass flask. Penetrating the thickness of a material which is investigated, e.g. packaging material, electrons affect all microbiologic objects which are on the walls and in the volume. This encompasses microbes, viruses, fungi, parasites, etc. At a critical dose of irradiation, all these microbiologic objects are destroyed.

In a case of the radio chemical sterilization, a different mechanism of sterilization occurs. One of the side results of interaction of the relativist electron beam with air is ionization of air and one of the consequences of it is ozone formation. When the irradiation object is air-tightly sealed (e.g. empty ampoules or ampoules with a medical preparation preserved or bottled food products, plastic-packages of food products) ozone forms under the effect of the electron penetration. This ozone interacts with microorganisms in the irradiation objects arid kills them owing to its (ozone's) toxicity. The high efficiency of this process is explained by that a sterilization-active ozone concentration persists for a few hours in the irradiated and air-tight packaged objects. Owing to this, a long-duration sterilization process is realized.

The virtue of the radiochemical sterilization technology is that it gives a possibility for sterilization of objects of large volumes by using relatively low energy electron beams (lower or 1 MeV beams). The shortcoming of it is the inability to sterilize non-air-tight packaged objects. As an example of them are: grain, flowing liquid products, large packages with a significant number of small objects, etc. In such cases it is better to use the method of “direct” electron sterilization.

Besides the effect of the electron energy, as it is known, the efficiency of electron sterilization depends also in the intensity of beams. It increases with the electron beam intensity. In the furthering of the efficiency there are certain technologic obstacles which are of the principle physical nature. These obstacles are related with the danger of partial or total destruction of the irradiation object by irradiation with an above the critical current density of electron beam. Traditionally this problem is solved by decreasing current density with a simultaneous increase of the irradiation time. However, this automatically leads to a significant decrease of the system productivity. These limitations interfere with an increase of the productivity of prototypes (in other words, an increase of productivity mass per unit time).

As it was mentioned above, for an increase of the productivity of the electron sterilizer in the proposed invention, structural versions are proposed in which the sterilization is achieved by a few subsequent electron beams. The performance of such sterilizers is illustrated on the examples FIG. 3 to FIG. 6. In the structure presented in FIG. 3, such possibility is enhanced by having a 3-channel MLIA 14. This means that during its performance this accelerator forms simultaneously three relativistic electron beams 10. Accordingly, the output device block 2 is made in the structural variant 11. Because of this, the structural block 15 of the output devices as well the scanning system 11 provide a simultaneous work with three independent electron beams 10. The principle, the work of this structure basically does not differ from the work of the sterilizer illustrated in FIG. 2. The difference is based on the movement process in the transport system 4, where the irradiation object 5 is sequentially irradiated first by the first, then by the second, and the third electron beam. Consequently, the duration of the exposition can be decreased (as compared with the one-beam system of FIG. 2) for that many times which is equal to the number of electron beams. Of course, the chosen number (3) of the electron beams is not essential. It depends on the demands of the project technology to a sterilizer.

Application of the multi-beam structure versions of MLIA opens a possibility for a more simple solution (than that shown on FIG. 3) of increase of sterilizer's productivity. The FIG. 4 shows a constructive idea of this solution. As it was mentioned before, the structural difference of this version from the version illustrated on FIG. 3 depends on the form of the irradiatio system block 3. In FIG. 4 it has a form of the electron-beam defocusing system 17 (e.g. defocusing magnetic lenses). In the presented structure, the electrons of each electron beam 18 move vertically down between the output from the four-channel MLIA 16 and the defocusing system 17. After the beam 18 passes the defocusing system 17, the trajectories of different electrons deviate from the vertical at different angles. This results in divergence of the initially linear electron beams 18. Consequently, the irradiation spot on the surface of the irradiated object 5 becomes bigger than in a case of not diverging beam irradiation. All of these irradiation spots merge into a continuous irradiation path 19. By a movement of the irradiation object 5 in the transport system 4, a parallel to itself movement of the irradiation path 19 takes place, resulting in a successive irradiation of the total surface of the object 5. At the same current density of electron beam, in this structural version, the irradiation-path width 12 appears much wider than the width of an analogous path in a one-beam system (look FIG. 2). Besides this, the principle of the performance of this structure version of the sterilizer does not differ from that of the previous examples.

The FIG. 5 illustrates the variant of the electron sterilizer structure with horizontal position of MLIA and the transport system 4. As it was mentioned, the peculiarity of this structure is that here the output system block 2 has a form of a turning (at right angle) magnetic system 22. Owing to this structure, the parallel horizontal electron beams form at the output of the working channels of MLIA 1 (here they are composed of accelerator sections 20 interconnected by transition devices 21) change the direction of their motion from horizontal to vertical under the effect of the magnetic fields of the turning system 22. As a result of it, a series of the vertically oriented electron beams are directed to the output of the irradiation system 3. Then, like in the previous structures, the latter are directed on the irradiation object 5, which in turn, moves on the horizontally-oriented transport system 4.

In the FIG. 6, the variant of the electron-sterilizer structure with the horizontal orientation of the MLIA 1 and the vertical orientation of the transport system 4, which has a form of a vertical transporter, pneumo-conductor, or turbo-conductor 25. As it was mentioned before, the peculiarity of this variant is the position of the irradiation system 3 directly near the wall of the vertically oriented transport system 25, in which the irradiation objects 5 (liquids, powders, etc.) are moved in the vertical direction. In all other action, the work principal of this variant resembles that of the structural variant which is presented in FIG. 5.

As it was mentioned before, in this invention, besides the structural versions in which the sterilization occurs as a direct result of the action of electron beams, a version is proposed in which the sterilization involves the effect of hard X-rays. The basic difference of the latter variants is in the structure of the irradiation-system. Simultaneously, the common peculiarity of such structural versions is that the electron beams never go beyond the limits of the vacuum system. This is achieved technologically by directing the electron beams toward the x-ray targets, which are located at the outlet of each of the irradiation system 3. Owing to the occurring effect of Bremsstrahlung, a fraction (8-10%) of the kinetic energy of the translational motion of electron beams transforms into the energy of hard X-ray irradiation.

The principle of the formation of a form of the X-ray-irradiation path on the surface of irradiation object 5 depends on the variant structure of the irradiation system 3. For instance, in a case when the irradiation system is structured as an X-ray target, the required form of the X-ray-beam irradiation is made by using a specific geometry of the X-ray target. In a different case, when the irradiation system has a form of scanning system, having an X-ray target at the outlet, the irradiating X-ray beam is formed as a scanning beam. This is realized technologically in the following way. Owing to scanning of electron beam, the angle of incidence of the beam on the X-ray target changes periodically. Finally, in the structures in which the irradiation system 3 holds a system of electron beam defocusing, the X-ray targets are positioned at the outlet of the system. Consequently, like in the previous case, the formed X-ray beam also diverges (defocuses). In all given cases, the sterilization of an irradiation object is achieved by the action of the hard X-ray irradiation on pathogenic bacteria, viruses, parasites, fungi, which are present in the objects of the irradiation treatment.

The invention can be used as a commercial-type compact electron sterilizer for sterilization of food products, medical and biologic preparations, medical and biologic equipments, disinfection of waters, including the waste waters, agricultural products (including grains and beans) etc. It is intended for destruction (or depression) of pathogenic bacteria, viruses, parasites and fungi which exist in the objects of treatment. Consequently, thus presented electron sterilizer corresponds to the patentability criterion “Industrial suitability”

1. The method for sterilizing materials comprising the steps of:
(a) providing a multi-channel linear induction accelerator system having an output of select electron beam energy and direction;
(b) providing an output assembly coupled in vacuum secure relationship with said linear induction accelerator system for transferring said output of select energy therefrom in a predetermined direction;
(c) manipulating said output from said output assembly to distribute it over a treatment region of controlled extent and with a distribution of output energy effective to non-destructively sterilize said material; and
(d) transporting said material through said treatment region.

2. The method of claim 1 in which:
said step (a) provides said multi-channel linear induction accelerator system as having a single channel with said output being present as a single beam; and
said step (c) manipulates said single beam by magnetically causing it to successively sweep across said treatment region.

3. The method of claim 2 in which said step (c) manipulates said output to provide a hard X-ray output.

4. The method of claim 1 in which:
said step (a) provides said multi-channel linear inductor accelerator system as having more than one channel, each providing a channel-designated discrete said output; and
said step (c) manipulates each said channel-designated output by magnetically causing it to sweep across that said treatment region associated with said channel-designated output.

5. The method of claim 4 in which said step (c) manipulates at least one said channel-designated output to provide a hard X-ray output.

6. The method of claim 1 in which:
said step (a) provides said multi-channel linear accelerator induction system as having more than one channel, each providing a channel-designated discrete said output; and
said step (c) manipulates each said channel-designated output by defocusing it to derive an expanded channel-designated output at said treatment region in a manner wherein said channel-designated outputs of adjacent said channels are caused to overlap and mutually extend over said treatment region.

7. The method of claim 6 in which said step (c) manipulates at least one said channel-designated output to provide a hard X-ray output.

8. The method of claim 6 in which:
said step (c) manipulates each said channel-designated output by azimuthally-symmetrically defocusing it.

9. The method of claim 6 in which:
said step (c) manipulates each said channel-designated output by azimuthally-asymmetrically defocusing it.

10. The method of claim 6 in which:
said step (a) provides said multi-channel linear induction accelerator system as having more than one channel, each said channel providing a channel designated discrete said output having a said select direction which is generally horizontal; and
said step (b) provides said output assembly as transferring said output in a said predetermined direction which is generally horizontal.

11. The method of claim 10 in which said step (c) manipulates at least one said channel-designated output to provide a hard X-ray output.

12. The method of claim 10 in which said step (c) manipulates each said channel-designated output by defocusing it to derive an expanded channel-designated output at said treatment region in a manner wherein said channel-designated output of adjacent said channels are caused to overlap and mutually extend over said treatment region.

13. The method of claim 12 in which:
said step (c) manipulates each said channel-designated output by azimuthally-symmetrically defocusing it.

14. The method of claim 12 in which:
said step (c) manipulates each said channel-designated output by azimuthally-asymmetrically defocusing it.

15. The method of claim 1 in which:
said step (a) provides said multi-channel linear induction accelerator system as having more than one channel, each said channel providing a channel-designated discrete said output having a said select direction which is generally horizontal;
said step (b) provides said output assembly with a said predetermined direction which is generally horizontal; and
said step (d) transports said material generally vertically through said treatment region.

16. The method of claim 15 in which said step (c) manipulates at least one said channel-designated output to provide a hard X-ray output.

17. The method of claim 15 in which said step (c) manipulates each said channel-designated output by defocusing it to derive an expanded channel-designated output at said treatment region in a manner wherein said channel-designated outputs of adjacent said channels are caused to overlap and mutually extend over said treatment region.

18. The method of claim 17 in which:
said step (c) manipulates each said channel-designated output by azimuthally-symmetrically defocusing it.

19. The method of claim 17 in which:
said step (c) manipulates each said channel-designated output by azimuthally-asymmetrically defocusing it.
